SEQUENCE LISTING (9 sequences)

SEQ ID NO: 1 - protein, 364 aa, Ganoderma applanatum
MFSKVFLSLVVLASSVAAAVPTVSRRATCTNGKTTANDACCVWFDVLDDIQENLFHGGQCGEDAHESLRLTFHDAIAFSPALTVAGQFGGGGADGSIIAHSDVELTYPVNDGLDEIIEASRPFAIKHNVSFGDFIQFAGAVGVANCNGGPQLSFFAGRSNDSQPSPPNLVPLPSDSADTILSRFSDAGFDALEVVWLLVSHTVGSQNTVDPSIAGAPFDSTPSDFDAQFFVETMLNGTLVPGDGLHDGQVNSPYPGEFRLQSDFALSRDSRTTCEWQKMIADRANMLQKFEVTMLKMSLLGFNQSALTDCSDVIPTATGTVQDPFIPAGLTVDDLQPACSSSAFPTVTTVAGAATSIPAVPLNS

SEQ ID NO: 2 - protein, 364 aa, Ganoderma applanatum
MFSKVFLSLVVLASSVAAAVPTVSRRATCTNGKTTANDACCVWFDVLDDIQENLFHGGQCGEDAHESLRLTFHDAIAFSPALTAAGQFGGGGADGSIIAHSDVELTYPVNDGLDEIVEASRPFAIKHNVSFGDFIQFAGAVGVANCNGGPQLSFFAGRSNDSQPSPPNLVPLPSDSADTILSRFSDAGFDALEVVWLLVSHTVGSQNTVDPSIPGAPFDSTPSDFDAQFFVETMLNGTLVPGDALHDGEVNSPYPGEFRLQSDFVLSRDSRTTCEWQKMIADRANMLQKFEVTMLKMSLLGFNQSALTDCSDVIPTATGTVQDPFIPAGLTVDDLQPACSSSAFPTVTIVAGTATSIPAVPLNS

SEQ ID NO: 3 - protein, 367 aa, Bjerkandera adusta
MAFKHLAAVLSIAFSLQAVQGAIIKRVACPDGRHTAINAACCNLFTVRDDIQRNMFDGGKCNDIAHQALRLTFHDAVAFSPALEAEGKFGGNGADGSIITFGNIETNFHPNIGLDEIVEIEKPFIARHNMTPGDFLHFAGAIAVTNCPGAPTISFSLGRPVATRPAPDGLVPEPFHTPDQIFARMLDALEFDPLETTWALIAHTVAAADDIDTSIPRSPFDSTPELFDGQFFIETQLKGTLFPGNGPNKGEVRSPLAGEMRLQSDFLIARDNRSACEWQSFGTDHDKLTNRFQFVLETLAMVGQDPTNMIDCSEVIPIPRNLTSAQIPHFPAGKTIRDVEAACPETPFPRLPTDAGRPTAVAPVPRG

SEQ ID NO: 4 - protein, 474 aa, Bjerkandera adusta
AKLGARQSRTTPLLTNFPGQAPLPSLEQHSTQRGANTLPLDNIQGDILVGMKKQKERFVFFHVNDATSFKTALKTYVPDHITSAQTLISDPSEQPLAFVNLAFSNTGLQALGVTDSLGDAQFPNGQFADASNLGDDLSQWVAPFTGTAIHGVFLIGSDQDSFLDQFENDISTAFGASITEVQALSGSARPGDLAGHEHFGFLDGISQPAVTGWETTVFPGQAVVPPGIILTGRDGDPTTRPSWALDGSFMAFRHFQQKVPEFNAYTLANAIPANSAGNLTQQEGAEFLGARMFGRWKSGAPIDLAPTADDPALGADPQRNNNFDFSDTLTDETKCPFAAHIRKTNPRQDLGGPVNTFHAIRSSIPYGPETSDAELASGVTSQDRGLLFVEYQSVIGNGFRFQQINWVNNAGFPFSKPIAPGIDPIIGQSPTRSTGGLDPLDQTKTFSVPLFVIPKGGEYFFMPSISALTSTIAA

SEQ ID NO: 5 - protein, 496 aa, Bjerkandera adusta
MRLSLFVVSVAVFIGSSTHVSAAKLGARQSRTTPLLTNFPGQAPLPSLEQHSTQRGANTLPLDNIQGDILVGMKKQKERFVFFHVNDATSFKTALKTYVPDHITSAQTLISDPSEQPLAFVNLAFSNTGLQALGVTDSLGDAQFPNGQFADASNLGDDLSQWVAPFTGTAIHGVFLIGSDQDSFLDQFENDISTAFGASITEVQALSGSARPGDLAGHEHFGFLDGISQPAVTGWETTVFPGQAVVPPGIILTGRDGDPTTRPSWALDGSFMAFRHFQQKVPEFNAYTLANAIPANSAGNLTQQEGAEFLGARMFGRWKSGAPIDLAPTADDPALGADPQRNNNFDFSDTLTDETKCPFAAHIRKTNPRQDLGGPVNTFHAIRSSIPYGPETSDAELASGVTSQDRGLLFVEYQSVIGNGFRFQQINWVNNAGFPFSKPIAPGIDPIIGQSPTRSTGGLDPLDQTKTFSVPLFVIPKGGEYFFMPSISALTSTIAA

SEQ ID NO: 6 - DNA, 1095 bp, Ganoderma applanatum
atgttctcaaaagtcttcctctccctcgtcgtcctcgcttcgtcggtcgccgccgctgtgccgacggtgagcagacgtgcgacgtgcaccaacgggaagaccaccgccaacgatgcgtgctgtgtctggttcgatgtcctcgacgacattcaagagaacctcttccatggtggacagtgcggggaagacgctcatgagtccctgaggctgactttccatgacgctatcgctttctctcctgctttgaccgtggcaggtcaattcggtggcggaggcgcggatggctcgatcattgcccactctgatgtcgagctgacatatcccgtcaacgacggtctggacgaaatcatcgaggcttcacgtccgtttgcaatcaagcacaacgtttccttcggtgacttcatccagttcgctggtgccgttggtgtcgcgaactgcaacggcggtccccagctctcgttcttcgctgggcggtccaacgactcgcagccgtcccctccgaacctcgtccctctcccgtcggactccgcggacacgattctctctcgcttcagcgacgcgggtttcgacgccctcgaggttgtgtggctcctggtctcccacaccgtcggttcgcagaacacggtcgacccaagcatcgccggcgcgcccttcgactcgacgccgtcggacttcgatgcccagttcttcgtcgagacgatgctcaacggcacactcgtccccggtgacggcctccatgacggccaagtgaactcaccctaccctggcgagttccgtctccagtccgacttcgcgctctctcgcgactcccggaccacctgcgagtggcaaaagatgatcgcggaccgtgcgaacatgctacagaagttcgaggtgacgatgctgaagatgtcgctgctcggcttcaatcagtcggcgctcaccgactgctccgacgtcatccccaccgcgaccggcacggtccaggatccgttcatccctgcgggcctgacggtggacgacctccagccggcgtgctcctcgtccgccttccctacggtgaccactgttgctggtgctgccacctctatccctgcggtgcccctgaactcctga

SEQ ID NO: 7 - DNA, 1095 bp, Ganoderma applanatum
atgttctcaaaagtcttcctctccctcgtcgtcctcgcttcgtcggtcgccgccgctgtgccgacggtgagcagacgtgcgacgtgcaccaacgggaagaccaccgccaacgatgcgtgctgtgtctggttcgatgtcctcgacgacattcaagagaacctcttccatggtggacagtgcggggaagacgctcatgagtccctgaggctgaccttccatgacgctatcgccttctctcctgctctgactgctgcaggtcaattcggtggcggtggcgctgatggctcgatcatcgcccactctgatgtcgagttgacatatcccgtcaacgacggcctggatgaaatcgtcgaagcttcccgtccgtttgcaatcaagcacaacgtctcgttcggtgacttcatccaattcgccggcgccgtcggggtggcgaactgcaatggcggccctcagctctcgttcttcgccggtcgctccaacgactcgcagccgtcccctccgaacctcgtccctctcccgtcggatagtgccgacacgatcctgtctcgcttcagcgatgcgggcttcgacgccctcgaggtcgtgtggctcctggtctcccataccgtcggttcgcagaacacggtcgatccgagcatccccggcgcgcctttcgactcgacgccgtcggacttcgatgcccagttcttcgtcgagacgatgctcaacggcacgctcgtccccggtgacgcgctccatgacggcgaagtgaactcgccctaccctggcgagttccgtctccagtccgactttgtgctctctcgcgactcccggaccacctgcgagtggcaaaagatgatcgcggaccgtgcgaacatgctacagaagttcgaggtgacgatgctgaagatgtcgctgctcggcttcaaccagtcggcgctcaccgactgctccgacgtcatccccaccgcgaccggcacggtccaggacccgttcatccccgcgggcctgacggtggacgacctccagccggcgtgctcatcgtctgccttccctacggtgaccattgttgctggtaccgccacttctattcctgctgtgccattgaactcctga

SEQ ID NO: 8 - DNA, 1104 bp, Bjerkandera adusta
atggccttcaagcacctcgccgctgtgctctctatcgccttctctcttcaggctgtccaaggcgcgatcatcaaacgcgtcgcctgtcccgacggcaggcacaccgcgatcaacgctgcgtgctgcaacctcttcacggtccgcgacgacatccagaggaacatgttcgacggtggcaaatgtaacgacatagctcaccaggccctccgtctcactttccatgacgccgttgctttctcgcccgcccttgaggctgagggcaaattcggtggtaacggtgcagacggctccatcatcaccttcggcaacattgagaccaacttccaccccaacatcggtctcgacgagatcgttgagattgagaagccgttcattgccaggcacaacatgacccccggagacttcttgcacttcgctggtgccattgctgttactaactgcccaggcgccccgaccatcagcttctccctcggacgccctgtggcgactcgtcctgcgcctgatggcctcgttcctgagccattccacacccccgaccaaatctttgctcgcatgttggacgccctggaatttgacccactcgagacgacttgggccttgattgcgcacactgttgctgccgccgacgatatcgacacatctattcctcggagccccttcgactcgactccggagctgtttgacggacagtttttcatcgagacgcagctcaagggcactttgttcccaggtaacggccccaacaagggagaggttcgctctcctcttgccggagagatgcgtcttcagtcggacttcctcatcgctcgtgacaaccgcagcgcttgcgagtggcaatccttcggcaccgaccacgacaaacttaccaaccgattccagttcgtccttgagactctcgccatggtcggccaggaccccacgaacatgatcgactgctccgaggtcatccccatcccccgcaacttgacttccgcgcagattcctcacttccccgctggaaagactatccgcgacgtcgaggctgcctgccctgaaactcccttccctcgcctccccaccgacgcaggccgccccacagctgtcgcccctgtcccccgcggataa

SEQ ID NO: 9 - DNA, 1491 bp, Bjerkandera adusta
atgcgtctttcgctgtttgtcgtgtcggtcgctgttttcatcgggtcgagcacgcatgtatctgctgctaaactcggtgcgaggcagtcgcgcacaacgcctctcctcacgaacttcccgggacaggccccgctgccttctctagagcaacactcgactcagaggggtgccaacaccctgcctctggataacatccagggcgacatcctggttggaatgaagaaacagaaggaacgcttcgtctttttccacgtaaatgacgcgacctcgttcaagacggcgttgaagacatacgtgcctgaccacatcacgtcggcccagacgttgatttcagacccctctgagcagccgctggctttcgtcaaccttgcgttctcgaacacgggtctccaggccctcggcgtcactgacagtctgggcgacgcgcaattccccaacggccagttcgccgacgcctcaaaccttggggacgacctcagccaatgggtggcgcctttcactggcaccgctatccatggtgtgttcttgattggtagtgatcaggattccttcctggatcagtttgagaacgatatctccaccgctttcggtgcctcgattactgaggtacaggcgctgagcgggtctgctcgtccaggagacctggcgggacatgaacacttcggtttcctcgacggcatctcgcagcccgcagtcacgggctgggagacgactgtcttccctggccaggcagttgtcccaccaggcatcatcctcactggacgtgacggcgacccgaccacccgtccttcatgggctctcgacggcagtttcatggcgttccgtcacttccaacagaaggtccccgagttcaacgcctacacgctggccaatgctatcccggccaacagcgcaggcaacctcactcagcaggagggtgccgagttcctcggtgcacgcatgtttggtcgctggaagagcggcgccccgatcgacctcgcacccacggcggacgatcccgcacttggtgctgacccccagaggaacaacaactttgacttctcggatacgctgacggacgagacgaagtgcccctttgctgcgcacatcaggaagacaaacccccgtcaggacttgggtggcccggttaacaccttccatgccatccgctccagtattccctacggcccagagacgtcggatgcagaacttgcgtcgggcgtgacttcgcaagaccgtggtcttctcttcgtcgagtaccaatccgtcatcggcaacggcttcaggttccaacagatcaactgggtcaacaatgcagggttcccattctccaagccaatcgcgcccggtattgaccccatcatcggccagtcgcctacacgctcgacgggcggcctcgaccccctcgaccagacgaagacgttcagcgtccctctgttcgtgatcccgaagggtggagagtacttcttcatgccctccatctctgcgctcacctccactatcgcggcttga

(19) United States
(12) Patent Application Publication
(10) Pub. No.: US 2017/0260483 A1
(43) Pub. Date: Sep. 14, 2017

(54) PEROXIDASES HAVING ACTIVITY FOR CAROTENOIDS

(71) Applicant: Henkel AG & Co. KGaA, Duesseldorf (DE)

(72) Inventors: Nina Mussmann, Willich (DE); Thomas Weber, Dormagen (DE); Timothy O'Connell, Landsberg am Lech (DE); Ralf G. Berger, Hannover (DE); Diana Linke, Rehburg (DE); Daniela Herbst, Duesseldorf (DE)

(73) Assignee: Henkel AG & Co. KGaA, Duesseldorf (DE)

(21) Appl. No.: 15/508,679

(22) PCT Filed: Sep. 8, 2015
(86) PCT No.: PCT/EP2015/070416

§ 371 Date: Mar. 3, 2017

(30) Foreign Application Priority Data
Sep. 11, 2014 (DE) 102014218229.8

(51) Int. Cl. C11D 3/386; C11D 11/00; C12N 9/08; C11D 3/39

(52) U.S. Cl. CPC C11D 3/38636; C11D 3/3942; C11D 11/0023; C11D 11/0017; C12N 9/0065; C12Y 111/01007

(57) ABSTRACT

A peroxidase, a method for producing the peroxidase, and an agent including at least one peroxidase are provided herein. The peroxidase includes an amino acid sequence that has a sequence identity of at least 60% to the amino acid sequence specified in SEQ ID NO:1 (Gap MnP1), across the entire length thereof; or has a sequence identity of at least 60% to the amino acid sequence specified in SEQ ID NO:2 (Gap MnP2), across the entire length thereof; or has a sequence identity of at least 80% to the amino acid sequence specified in SEQ ID NO:3 (Bja LiP), across the entire length thereof; or has a sequence identity of at least 60% to the amino acid sequence specified in SEQ ID NO:4 (Bja DyP), across the entire length thereof.

### CROSS-REFERENCE TO RELATED APPLICATION

This application is a U.S. National-Stage entry under 35 U.S.C. §371 based on International Application No. PCT/EP2015/070416, filed Sep. 8, 2015, which was published under PCT Article 21(2) and which claims priority to German Application No. 10 2014 218 229.8, filed Sep. 11, 2014, which are all hereby incorporated in their entirety by reference.

### TECHNICAL FIELD

The present disclosure lies in the field of enzyme technology. The disclosure relates to peroxidases having activity for carotenoids, to the production thereof, to all sufficiently similar peroxidases and to nucleic acids coding therefor, and also to host organisms which contain said nucleic acids. The disclosure also relates to methods which use said peroxidases, and to agents containing them, in particular washing and cleaning agents.

### BACKGROUND

The use of enzymes in washing and cleaning agents is established in the prior art. They are used to expand the performance spectrum of the agents in question according to their special activities. These include in particular hydrolytic enzymes such as proteases, amylases, lipases and cellulases. The first three aforementioned enzymes hydrolyze proteins, starches and fats and therefore contribute directly to soil removal. Cellulases are used in particular because of their effect on fabrics. To increase the bleaching effect, however, oxidoreductases, for example oxidases, oxygenases, catalases (which react as peroxidase at low H₂O₂ concentrations), peroxidases such as haloperoxidase, chloroperoxidase, bromoperoxidase, lignin peroxidase, glucose peroxidase or manganese peroxidase, dioxygenases or laccases (phenol oxidases, polyphenol oxidases) are also used in the washing and cleaning agents.

Suitable enzymatic bleaching systems are known in the prior art, for example from the international patent publications WO 98/45398 A1, WO 2004/058955 A2, WO 2005/124012 and WO 2005/056782 A2. Such enzymatic systems can advantageously be combined with organic, particularly preferably aromatic, compounds which interact with the enzymes in order to enhance the activity of the oxidoreductases in question (enhancers) or to ensure the electron flow in the case of very different redox potentials between the oxidizing enzymes and the soil (mediators).

Conventional bleaching systems based on percarbonate, peroxide or chlorine cannot be used in water-containing formulations, that is to say in particular many liquid formulations. Moreover, the use of such systems is perceived by consumers as aggressive and harmful to the environment in comparison to enzymatic systems. In this respect, the use of enzymatic systems is desirable for reasons of sustainability.

However, enzymatic bleaching systems are usually also based on the enzymatic generation of hydrogen peroxide by the breakdown of suitable enzyme substrates. These substrates have to be added to the washing or cleaning agents and represent an additional cost factor and in some cases also an additional toxicological or allergological risk factor. In liquid one-component systems, there is also the problem that the substrate and the enzyme come into contact even before use in the washing or cleaning liquor, and therefore a premature breakdown of the substrate must be avoided at great effort.

Bleaching systems are necessary, however, for removing certain highly staining soils on textiles and hard surfaces, for example carotenoid-containing soils, in order to achieve a satisfactory cleaning performance. Carotenoid-containing soils on textiles are difficult to remove with conventional liquid washing agents. Moreover, carotenoid-containing soils on dishes pose the problem that they are distributed in the cleaning liquor in automatic dishwashing and diffuse into plastics and discolor the latter. These discolorations are familiar to the user and it is desirable to reduce this phenomenon.

There is therefore a need for substrate-independent enzymatic systems which have a lightening effect particularly on carotenoid-containing soils.

In order to be suitable for use in washing and cleaning agents, it is also desirable that such enzyme systems have an enzymatic activity in the neutral to slightly alkaline pH range and in a broad temperature range up to 95° C., in particular in the range 30-55° C.

### BRIEF SUMMARY

A peroxidase, a method for producing the peroxidase, and an agent including at least one peroxidase are provided herein. In one embodiment, the peroxidase includes an amino acid sequence that has a sequence identity of at least 60% to the amino acid sequence specified in SEQ ID NO:1 (Gap MnP1), across the entire length thereof; or has a sequence identity of at least 60% to the amino acid sequence specified in SEQ ID NO:2 (Gap MnP2), across the entire length thereof; or has a sequence identity of at least 80% to the amino acid sequence specified in SEQ ID NO:3 (Bja LiP), across the entire length thereof; or has a sequence identity of at least 60% to the amino acid sequence specified in SEQ ID NO:4 (Bja DyP), across the entire length thereof.

In another embodiment, the method includes culturing a host cell which includes the peroxidase. The method further includes isolating the peroxidase from the culture medium or from the host cell.

In another embodiment, the agent includes at least one peroxidase. The peroxidase includes an amino acid sequence that has a sequence identity of at least 60% to the amino acid sequence specified in SEQ ID NO:1 (Gap MnP1), across the entire length thereof; or has a sequence identity of at least 60% to the amino acid sequence specified in SEQ ID NO:2 (Gap MnP2), across the entire length thereof; or has a sequence identity of at least 80% to the amino acid sequence specified in SEQ ID NO:3 (Bja LiP), across the entire length thereof; or has a sequence identity of at least 60% to the amino acid sequence specified in SEQ ID NO:4 (Bja DyP), across the entire length thereof.

### DETAILED DESCRIPTION

The following Detailed Description is merely exemplary in nature and is not intended to limit the various embodiments or the application and uses thereof. Furthermore, there is no intention to be bound by any theory presented in the preceding background or the following detailed description.

Peroxidases from Bjerkandera adusta and Ganoderma applanatum have now been found, which have the desired properties and therefore are particularly suitable for use in washing and cleaning agents. The peroxidases of fungal origin that have been found have a marked activity on carotenoids. As a result, the enzyme can be used without additional substrates for lightening carotenoid-containing soils.

In a first aspect, the disclosure therefore relates to a peroxidase comprising an amino acid sequence that has a sequence identity of at least 60%, preferably at least 70%, to the amino acid sequence specified in one of SEQ ID NO:1 (Gap MnP1), across the entire length thereof; or
(i) has a sequence identity of at least 60%, preferably at least 70%, to the amino acid sequence specified in one of SEQ ID NO:2 (Gap MnP2), across the entire length thereof; or
(ii) has a sequence identity of at least 80%, preferably at least 90%, to the amino acid sequence specified in SEQ ID NO:3 (Bja LiP), across the entire length thereof; or
(iii) has a sequence identity of at least 60%, preferably at least 70%, to the amino acid sequence specified in SEQ ID NO:4 (Bja DyP), across the entire length thereof.

In different embodiments, the peroxidase has an enzymatic activity for carotenoids. The enzymes described herein are preferably of fungal origin, in particular homologs of the Ganoderma applanatum manganese peroxidases having the amino acid sequences specified in SEQ ID Nos. 1 and 2, of the Bjerkandera adusta lignin peroxidase having the amino acid sequence specified in SEQ ID NO:3, or of the Bjerkandera adusta peroxidase having the amino acid sequence specified in SEQ ID NO:4.

The peroxidases described herein have an enzymatic activity, that is to say they are capable of oxidatively cleaving suitable enzyme substrates, in particular carotenoids. The oxidative cleavage is independent of the presence of hydrogen peroxide. A peroxidase described herein is preferably a mature peroxidase, that is to say the catalytically active molecule without signal peptide(s) and/or propeptide(s). Unless otherwise stated, the specified sequences also refer to mature enzymes in each case. By way of example, the mature peroxidase without signal peptide from Bjerkandera adusta has the amino acid sequence specified in SEQ ID NO:4, while the amino acid sequence of the same enzyme with an N-terminal signal peptide having a length of 22 amino acids is specified in SEQ ID NO:5.

The term “carotenoids”, as used herein, denotes compounds from the substance class of the terpenes, which occur as natural pigments producing a yellow to reddish color. About 800 different carotenoids are known, which occur primarily in the chromoplasts and plastids of plants, in bacteria, but also in the skin, the shell, and in the carapace of animals and in the feathers and in the egg yolk of birds, if the animals in question consume pigment-containing plant material with their food. Only bacteria, plants and fungi are capable of synthesizing these pigments de novo. Carotenoids are formally made up of 8 isoprene units and therefore are considered to be tetraterpenes. They are divided into carotenes, which are made up of only carbon and hydrogen, and xanthophylls, which are oxygen-containing derivatives of the carotenes. The absorption spectrum of the carotenoids occurs at wavelengths in the range from 400 to 500 nanometers. The best-known and most frequently occurring carotenoid is β-carotene (carrot), which is also known as provitamin A. Other frequently occurring carotenoids are α-carotene, lycopene (tomato), β-cryptoxanthin, capsanthin (red paprika), lutein and zeaxanthin.

Preferred embodiments of the peroxidases also have a particular stability in washing or cleaning agents, for example with respect to surfactants and/or bleaching agents and/or with respect to temperature effects, in particular with respect to high temperatures, for example between 50 and 65° C., in particular 60° C., and/or with respect to acidic or alkaline conditions and/or with respect to changes in pH and/or with respect to denaturing or oxidizing agents and/or with respect to proteolytic degradation and/or with respect to a change in the redox conditions.

In different embodiments of the disclosure, the peroxidases comprise an amino acid sequence that
(i) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.5%, 99.8% or 100% identical to the amino acid sequence specified in SEQ ID NO:1, across the entire length thereof; or
(ii) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.5%, 99.8% or 100% identical to the amino acid sequence specified in SEQ ID NO:2, across the entire length thereof; or
(iii) is at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.5%, 99.8% or 100% identical to the amino acid sequence specified in SEQ ID NO:3, across the entire length thereof; or
(iv) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.5%, 99.8% or 100% identical to the amino acid sequence specified in SEQ ID NO:4, across the entire length thereof.

Numerical values which are specified herein without decimal places refer in each case to the full specified value with one decimal place. For example, “99%” stands for “99.0%”.

Numerical values which are specified herein without decimal places refer in each case to the full specified value with one decimal place.

The term “approximately” in connection with a numerical value refers to a variation of ±10% with regard to the specified numerical value.

In a further aspect, the disclosure relates to an agent which is characterized in that it contains a peroxidase comprising an amino acid sequence that
(i) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.5%, 99.8% or 100% identical to the amino acid sequence specified in SEQ ID NO:1, across the entire length thereof; or
(ii) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.5%, 99.8% or 100% identical to the amino acid sequence specified in SEQ ID NO:2, across the entire length thereof; or
(iii) is at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.5%, 99.8% or 100% identical to the amino acid sequence specified in SEQ ID NO:3, across the entire length thereof; or
(iv) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.5%, 99.8% or 100% identical to the amino acid sequence specified in SEQ ID NO:4, across the entire length thereof.

Such a peroxidase advantageously has an enzymatic activity for carotenoids, that is to say is capable of using carotenoids as a substrate and of oxidatively cleaving carotenoids. The activity for carotenoids is demonstrable since it can be measured for example using the assays described in the examples and preferably is also quantifiable.

The enzymes used in the agent are preferably of fungal origin, in particular from Basidiomycota, particularly preferably homologs of the Ganoderma applanatum or Bjerkandera adusta peroxidases having the amino acid sequence specified in SEQ ID Nos. 1-4. The agent is preferably a washing or cleaning agent, including an (automatic) dishwashing detergent. Since peroxidases described herein have advantageous cleaning performances particularly on carotenoid-containing soils, the agents are particularly suitable and advantageous for removing such carotenoid-containing soils. Such agents contain the peroxidases described herein in an amount from about 1×10⁻⁸ to about 1% by weight, about 1×10⁻⁷ to about 0.5% by weight, from about 0.00001 to about 0.3% by weight, from about 0.0001 to about 0.2% by weight, and particularly preferably from about 0.001 to about 0.1% by weight % by weight, in each case based on active protein.

The (active) protein concentration can be determined by means of known methods, for example the BCA method (bicinchoninic acid; 2,2′-biquinolyl-4,4′-dicarboxylic acid) or the Biuret method.

The identity of nucleic acid or amino acid sequences is determined by a sequence comparison. This sequence comparison is based on the typically used BLAST algorithm, which is established in the prior art (cf. for example Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, pp. 3389-3402) and is carried out basically in that similar sequences of nucleotides or amino acids in the nucleic acid or amino acid sequences are matched to one another. A tabular matching of the positions in question is called an alignment. Another algorithm available in the prior art is the FASTA algorithm. Sequence comparisons (alignments), in particular multiple sequence comparisons, are compiled using computer programs. For example, use is frequently made of the Clustal series (cf. for example Chenna et al. (2003): Multiple sequence alignment with the Clustal series of programs. Nucleic Acid Research 31, 3497-3500, and Larkin et al. Bioinformatics, 23, 2947-2948), T-Coffee (cf. for example Notredame et al. (2000): T-Coffee: A novel method for multiple sequence alignments. J. Mol. Biol. 302, 205-217), or programs based on these programs or algorithms. In the present patent application, all sequence comparisons (alignments) were created using the computer program Vector NTI® Suite 10.3 (Invitrogen Corporation, 1600 Faraday Avenue, Carlsbad, Calif., USA) with the predefined standard parameters, whose AlignX module for the sequence comparisons is based on ClustalW.

Such a comparison also permits a conclusion on the similarity of the compared sequences. It is usually given as a percent identity, that is to say the proportion of identical nucleotides or amino acid residues at the same positions or at positions corresponding to one another in an alignment. In the case of amino acid sequences, the more broadly construed term of homology includes conserved amino acid exchanges, that is to say amino acids with similar chemical activity, since these perform mostly similar chemical activities within the protein. The similarity of compared sequences can therefore also be given as a percent homology or percent similarity. Indications of identity and/or homology can be given for entire polypeptides or genes or only for individual regions. Homologous or identical regions of various nucleic acid or amino acid sequences are therefore defined by matches in the sequences. Such regions often have identical functions. They may be small and comprise only a few nucleotides or amino acids. Often such small regions carry out functions essential for the overall activity of the protein. It can therefore be useful to relate sequence matches only to individual, optionally small regions. However, unless otherwise stated, the identity or homology data in the present application relate to the entire length of the nucleic acid or amino acid sequence specified in each case.

The peroxidases described herein may have amino acid modifications, in particular amino acid substitutions, insertions or deletions, compared to the sequences specified in SEQ ID Nos. 1-4. Such peroxidases are further developed for example by targeted genetic modification, that is to say by mutagenesis methods, and are optimized for particular use purposes or with regard to specific properties (for example with regard to their catalytic activity, stability, etc.). Furthermore, nucleic acids described herein can be introduced into recombination batches and thereby used to generate completely novel peroxidases or other polypeptides.

The aim is to introduce targeted mutations, such as substitutions, insertions or deletions, into the molecules in order to improve for example the performance of the enzymes described herein. To this end, it is possible to modify in particular the surface charges and/or the isoelectric point of the molecules and thus the interactions thereof with the substrate. For example, the net charge of the enzymes can be modified in order thereby to influence the substrate bonding, in particular for use in washing and cleaning agents. As an alternative or in addition, the stability of the peroxidases can be increased by one or more appropriate mutations and the performance thereof can be improved as a result.

A further subject matter of the disclosure is therefore a peroxidase which is characterized in that it can be obtained from a peroxidase as described above as the starting molecule by single or multiple conservative amino acid substitution. The term “conservative amino acid substitution” means the exchange (substitution) of one amino acid residue for another amino acid residue, wherein this exchange does not lead to a change in the polarity or charge at the position of the exchanged amino acid, for example the exchange of one non-polar amino acid residue for another non-polar amino acid residue. Conservative amino acid substitutions in the context of the disclosure comprise for example: G=A=S, I=V=L=M, D=E, N=Q, K=R, Y=F, S=T, G=A=I=V=L=M=Y=F=W=P=S=T.

As an alternative or in addition, the peroxidase is characterized in that it can be obtained from a peroxidase described herein as the starting molecule by fragmentation, deletion mutagenesis, insertion mutagenesis or substitution mutagenesis and comprises an amino acid sequence that (i) matches the starting molecule having the amino acid sequence according to one of SEQ ID Nos. 1-3 over a length of at least 100, 150, 200, 250, 300, 310, 320, 330, 340, 350 or 360 contiguous amino acids, or (ii) matches the starting molecule having the amino acid sequence according to SEQ ID NO:4 over a length of at least 100, 150, 200, 250, 300, 310, 320, 330, 340, 350, 360, 370, 380, 390, 400, 410, 420, 430, 440, 450, 460, 470, 480 or 490 contiguous amino acids.

It is thus possible for example to delete other individual amino acids at the termini or in the loops of the enzyme, without the enzymatic activity being lost or reduced as a result. Furthermore, by virtue of such fragmentation, deletion mutagenesis, insertion mutagenesis or substitution mutagenesis, for example the allergenicity of the enzymes in question can also be reduced and thus the usability thereof can be improved overall. Advantageously, the enzymes retain their enzymatic activity even after mutagenesis, that is to say their enzymatic activity corresponds at least to that of the starting enzyme, that is to say in one preferred embodiment the enzymatic activity is at least 80%, preferably at least 90% of the activity of the starting enzyme. Substitutions can also exhibit advantageous effects. Both individual and multiple contiguous amino acids can be exchanged for other amino acids.

A further subject matter of the disclosure is a previously described peroxidase which is additionally stabilized, in particular by one or more mutations, for example substitutions, or by coupling to a polymer. This is because an increase in the stability during storage and/or during use, for example during the washing process, has the result that the enzymatic activity lasts longer and thus the cleaning performance is improved. In principle, all stabilization options which are expedient and/or described in the prior art may be used. Preference is given to those stabilizations which are achieved by mutations of the enzyme itself, since such stabilizations require no further work steps after the obtaining of the enzyme.

Further options for stabilization are for example:
modifying the binding of metal ions or cofactors, for example by exchanging one or more of the amino acid(s) involved in the binding for one or more other amino acids;
protecting against the effect of denaturing agents, such as surfactants, by mutations which cause a change in the amino acid sequence on or at the surface of the protein;
exchanging amino acids situated close to the N-terminus for those which come into contact with the rest of the molecule presumably via non-covalent interactions and thus contribute to the retention of the globular structure.

Preferred embodiments are those in which the enzyme is stabilized in multiple ways, since multiple stabilizing mutations have an additive or synergistic effect. The enzymes described herein may contain manganese ions as cofactors and thus are stabilized variants, inter alia those in which the binding of the manganese has been modified.

A further subject matter of the disclosure is a peroxidase as described above which is characterized in that it has at least one chemical modification. A peroxidase having such a modification is referred to as a derivative, that is to say the peroxidase is derivatized.

In the context of the present application, derivatives will be understood to mean those proteins whose pure amino acid chain has been chemically modified. Such derivatizations can be performed for example in vivo by the host cell that expresses the protein. Linkages of low-molecular-weight compounds, such as of lipids or oligosaccharides, are to be emphasized in particular in this regard. However, derivatizations can also be carried out in vitro, for instance by chemical conversion of a side chain of an amino acid or by covalent bonding of a different compound to the protein. The linkage of amines to carboxyl groups of an enzyme in order to modify the isoelectric point is possible for example. Another such compound can also be a further protein that is bound for example via bifunctional chemical bonds to a protein described herein. Derivatization is likewise to be understood as covalent bonding to a macromolecular carrier, or also as a non-covalent inclusion into suitable macromolecular cage structures. Derivatizations can for example influence the substrate specificity or strength of bonding to the substrate, or can bring about a temporary blockage of enzymatic activity if the linked-on substance is an inhibitor. This can be useful for example for the period of storage. Such modifications can furthermore influence the stability or the enzymatic activity. They can moreover also serve to decrease the allergenicity and/or immunogenicity of the protein and thus for example to increase the skin compatibility thereof. By way of example, linkages to macromolecular compounds, for example polyethylene glycol, can improve the protein with regard to stability and/or skin compatibility.

Derivatives of a protein described herein can also be understood in the broadest sense as preparations of said proteins. Depending on the extraction, processing or preparation, a protein can be associated with various other substances, for example from the culture of the producing microorganisms. A protein can also have had other substances added to it in a targeted manner, for example in order to increase the storage stability thereof. For this reason, all preparations of a protein described herein are also included. This is also irrespective of whether or not it actually displays this enzymatic activity in a specific preparation. This is because it may be desirable for it to possess little or no activity during storage and to perform its enzymatic function only at the time of use. This can be controlled for example by suitable accompanying substances.

The present disclosure encompasses the above-described peroxidases and variants and derivatives thereof both as such and also as a component of an agent, in particular a washing and cleaning agent, as defined above.

A further subject matter of the disclosure is a nucleic acid that codes for a peroxidase described herein, in particular a nucleic acid which comprises one of the nucleotide sequences specified in SEQ ID Nos. 6-9, and also a vector containing such a nucleic acid, in particular a cloning vector or an expression vector.

These can be DNA or RNA molecules. They can exist as a single strand, as a single strand complementary to said single strand, or as a double strand. In the case of DNA molecules in particular, the sequences of both complementary strands in all three possible reading frames are to be considered in each case. Account must also be taken of the fact that different codons, that is to say base triplets, can code for the same amino acids, so that a given amino acid sequence can be coded by a plurality of different nucleic acids. Because of this degeneracy of the genetic code, all nucleic acid sequences that can encode one of the above-described peroxidases are included in this subject matter of the disclosure. A person skilled in the art is capable of unequivocally determining these nucleic acid sequences since, despite the degeneracy of the genetic code, defined amino acids are to be associated with individual codons. A person skilled in the art, proceeding from an amino acid sequence, can therefore readily ascertain nucleic acids coding for said amino acid sequence. Furthermore, in the case of nucleic acids described herein, one or more codons can be replaced by synonymous codons. This aspect refers in particular to the heterologous expression of the enzymes described herein. Each organism, for example a host cell of a production strain, thus has a specific codon usage. Codon usage will be understood to mean the translation of the genetic code into amino acids by the respective organism. Bottlenecks in protein biosynthesis can occur if the codons located on the nucleic acid are faced in the organism with a comparatively small number of loaded tRNA molecules. Although it codes for the same amino acid, the result is that a codon is translated less efficiently in the organism than a synonymous codon coding for the same amino acid. Because of the presence of a greater number of tRNA molecules for the synonymous codon, the latter can be translated more efficiently in the organism. Accordingly, the present disclosure also encompasses those nucleotide sequences that are codon-optimized for expression in a particular host organism. The sequence identity in this regard can be low in comparison with the original, but the coded protein nevertheless remains identical.

Using methods commonly known today, such as for example chemical synthesis or the polymerase chain reaction (PCR) in combination with standard methods of molecular biology and/or protein chemistry, a person skilled in the art is capable of preparing, on the basis of known DNA sequences and/or amino acid sequences, the corresponding nucleic acids up to complete genes. Such methods are known for example from Sambrook, J., Fritsch, E. F. and Maniatis, T. 2001. Molecular cloning: a laboratory manual, 3rd edition, Cold Spring Laboratory Press.

In the context of the present disclosure, vectors will be understood to mean elements which are made up of nucleic acids and which contain a nucleic acid described herein as a characterizing nucleic acid region. They make it possible to establish said nucleic acid as a stable genetic element in a species or a cell line over multiple generations or cell divisions. Particularly when used in bacteria, vectors are special plasmids, that is to say circular genetic elements. In the context of the present disclosure, a nucleic acid described herein is cloned into a vector. The vectors include for example those originating from bacterial plasmids, viruses or bacteriophages, or predominantly synthetic vectors or plasmids having elements of very diverse origin. With the further genetic elements present in each case, vectors are capable of establishing themselves as stable units in the relevant host cells over multiple generations. They can be present extrachromosomally as separate units or be integrated into a chromosome or into chromosomal DNA.

Expression vectors comprise nucleic acid sequences that enable them to replicate in the host cells containing them, preferably microorganisms, particularly preferably bacteria, and to express a nucleic acid contained therein. The expression is influenced in particular by the promoter(s) that regulate transcription. In principle, the expression can occur by the natural promoter, originally localized before the nucleic acid to be expressed, but also by a host cell promoter provided on the expression vector or by a modified or completely different promoter of a different organism or a different host cell. In the present case, at least one promoter is provided for the expression of a nucleic acid described herein and used for the expression thereof. Expression vectors can furthermore be regulatable, for example changing the culturing conditions or when the host cells containing them reach a specific cell density, or by adding specific substances, in particular activators of gene expression. One example of such a substance is the galactose derivative isopropyl-β-D-thiogalactopyranoside (IPTG), which is used as an activator of the bacterial lactose operon (lac operon). In contrast to expression vectors, the contained nucleic acid is not expressed in cloning vectors.

A further subject matter of the disclosure is a non-human host cell which contains a nucleic acid described herein or a vector described herein, or which contains a peroxidase described herein, in particular one which secretes the peroxidase into the medium surrounding the host cell. A nucleic acid described herein or a vector described herein is preferably transformed into a microorganism, which then represents a host cell. The nucleic acid described herein is preferably heterologous in regard to the host organism, that is to say is not a sequence occurring naturally in the host organism. Alternatively, individual components, that is to say nucleic acid parts or fragments of a nucleic acid described herein, can be also be introduced into a host cell in such a way that the resulting host cell contains a nucleic acid described herein or a vector described herein. This procedure is particularly suitable when the host cell already contains one or more constituents of a nucleic acid described herein or of a vector described herein, and the further constituents are then correspondingly supplemented. Cell transformation methods are established in the prior art and are sufficiently known to the person skilled in the art. Suitable host cells are in principle any cells, that is to say prokaryotic or eukaryotic cells. Preference is given to those host cells which can be advantageously genetically manipulated, for example as regards the transformation using the nucleic acid or vector and the stable establishment thereof, for example single-cell fungi or bacteria. Preferred host cells are also notable for being readily manipulated in microbiological and biotechnological terms. This refers for example to easy culturability, high growth rates, low demands for fermentation media, and good production and secretion rates for foreign proteins. Preferred host cells described herein secrete the (transgenically) expressed protein into the medium surrounding the host cells. Furthermore, after being produced, the peroxidases can be modified by the cells producing them, for example by the addition of sugar molecules, formylations, aminations, etc. Such post-translational modifications can functionally influence the peroxidase.

Further preferred embodiments are represented by those host cells whose activity can be regulated on the basis of genetic regulation elements that are provided for example on the vector, but can also be present at the outset in these cells. They can be stimulated to expression for example by the controlled addition of chemical compounds serving as activators, by modifying the culturing conditions, or when a specific cell density is reached. This allows an inexpensive production of the proteins described herein. One example of such a compound is IPTG, as described above.

Host cells can be prokaryotic or bacterial cells. Bacteria are notable for short generation times and few demands in terms of culturing conditions. As a result, cost-effective culturing methods or production methods can be established. In addition, the person skilled in the art has extensive experience with bacteria in fermentation technology. Gram-negative or Gram-positive bacteria may be suitable for a specific production, for various reasons to be determined experimentally in the individual case, such as nutrient sources, product formation rate, time requirement, etc.

In Gram-negative bacteria, such as for example Escherichia coli, a plurality of proteins are secreted into the periplasmic space, that is to say into the compartment between the two membranes enclosing the cell. This can be advantageous for specific applications. Furthermore, Gram-negative bacteria can also be configured so that they discharge the expressed proteins not only into the periplasmic space but also into the medium surrounding the bacterium. Gram-positive bacteria on the other hand, such as for example bacilli or actinomycetes, or other representatives of the Actinomycetales, possess no external membrane so that secreted proteins are delivered immediately into the medium, usually the nutrient medium, surrounding the bacteria, from which medium the expressed proteins can be purified. They can be isolated directly from the medium or processed further. In addition, Gram-positive bacteria are related or identical to most source organisms for technically important enzymes, and usually themselves form comparable enzymes, so that they possess similar codon usage and their protein synthesis apparatus is naturally correspondingly directed.

Host cells described herein can be modified in terms of their requirements for culture conditions, can comprise other or additional selection markers, or can also express other or additional proteins. They can also be, in particular, host cells that transgenically express multiple proteins or enzymes.

The present disclosure is in principle applicable to all microorganisms, in particular to all fermentable microorganisms, and has the result that proteins described herein can be produced by the use of such microorganisms. Such microorganisms then represent host cells in the context of the disclosure.

In a further embodiment of the disclosure, the host cell is characterized in that it is a bacterium, preferably one selected from the group of the genera Escherichia, Klebsiella, Bacillus, Staphylococcus, Corynebacterium, Arthrobacter, Streptomyces, Stenotrophomonas and Pseudomonas, more preferably one selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

However, the host cell may also be a eukaryotic cell which is characterized in that it possesses a cell nucleus. A further subject matter of the disclosure is therefore a host cell which is characterized in that it possesses a cell nucleus. In contrast to prokaryotic cells, eukaryotic cells are capable of post-translationally modifying the formed protein. Examples thereof are fungi such as basidiomycetes, actinomycetes, or yeasts such as Saccharomyces or Kluyveromyces. This may be particularly advantageous for example if the proteins are to undergo specific modifications, enabled by such systems, in connection with their synthesis. Modifications that eukaryotic systems carry out particularly in conjunction with protein synthesis include for example the bonding of low-molecular-weight compounds such as membrane anchors or oligosaccharides. Such oligosaccharide modifications can be desirable for example in order to lower the allergenicity of an expressed protein. Co-expression with the enzymes naturally formed by such cells, such as for example cellulases or lipases, can also be advantageous. Thermophilic fungal expression systems, for example, can furthermore be particularly suitable for the expression of temperature-resistant proteins or variants. Fungal expression systems are preferred in the context of the disclosure.

The host cells are cultured and fermented in a conventional manner, for example in discontinuous or continuous systems. In the former case, a suitable nutrient medium is inoculated with the host cells, and the product is harvested from the medium after a period of time to be determined experimentally. Continuous fermentations are notable for the achievement of a flow equilibrium in which, over a comparatively long time period, cells die off in part but also regrow, and the formed protein can be removed simultaneously from the medium.

The host cells described herein are preferably used to produce the peroxidases described herein. A further subject matter of the disclosure is therefore a method for producing a peroxidase, which method comprises

a) culturing a host cell described herein
b) isolating the peroxidase from the culture medium or from the host cell.

This subject matter of the disclosure preferably comprises fermentation methods. Fermentation methods are known per se from the prior art and represent the actual industrial-scale production step, generally followed by a suitable purification method for the produced product, for example the peroxidase described herein. All fermentation methods based on a suitable method for producing a peroxidase described herein represent embodiments of this subject matter of the disclosure.

Fermentation methods which are characterized in that fermentation is carried out via an inflow strategy are particularly appropriate. In this case, the media constituents consumed during continuous culturing are fed in. Considerable increases both in cell density and in cell mass or dry mass and/or especially in the activity of the peroxidase of interest can be achieved in this way. Furthermore, the fermentation can also be configured so that undesirable metabolic products are filtered out or are neutralized by the addition of a buffer or suitable counterions.

The peroxidase produced can be harvested from the fermentation medium. A fermentation method of this kind is preferred over isolation of the peroxidase from the host cell, that is to say product preparation from the cell mass (dry mass), but requires the provision of suitable host cells or one or more suitable secretion markers or mechanisms and/or transport systems, so that the host cells secrete the peroxidase into the fermentation medium. Alternatively, without secretion, the peroxidase can be isolated from the host cell, that is to say purification thereof from the cell mass, for example by precipitation using ammonium sulfate or ethanol, or by chromatographic purification.

All the above facts can be combined to form methods for producing peroxidases described herein.

In the agents described herein, in particular washing and cleaning agents, the enzymes to be used can be formulated together with accompanying substances, for instance from the fermentation, or with stabilizers. In liquid formulations, the enzymes are preferably used as liquid enzyme formulation(s).

The peroxidases can be protected, particularly during storage, against damage such as for example inactivation, denaturation or decomposition, for instance due to physical influences, oxidation or proteolytic cleavage. Inhibition of proteolysis is particularly preferred in microbial production. The described agents may contain stabilizers for this purpose.

Peroxidases with cleaning activity are generally not provided in the form of the pure protein but rather in the form of stabilized, storable and transportable formulations. These ready-made formulations include for example the solid preparations obtained by granulation, extrusion or lyophilization or, particularly in the case of liquid or gel-like agents, solutions of the enzymes, advantageously as concentrated as possible, low in water, and/or combined with stabilizers or other auxiliaries.

Alternatively, the enzymes may be encapsulated both for solid and liquid delivery forms, for example by spray-drying or extrusion of the enzyme solution together with a preferably natural polymer or in the form of capsules, for example those in which the enzymes are enclosed in a solidified gel, or in those of the core-shell type, in which an enzyme-containing core is coated with a water-impermeable, air-impermeable and/or chemical-impermeable protective layer. In addition, further active substances, for example stabilizers, emulsifiers, pigments, bleaches or colorants, can be applied in deposited layers. Such capsules are applied by methods known per se, for example by agitated or roll granulation or in fluidized bed processes. Advantageously, such granules are low-dusting, for example due to application of polymeric film formers, and storage-stable as a result of said coating.

It is also possible to formulate two or more enzymes together, so that a single granule has multiple enzymatic activities.

As is clear from the preceding explanations, the enzyme protein constitutes only a fraction of the total weight of conventional enzyme preparations. Preferably used peroxidase preparations contain between about 0.1 and about 40% by weight, preferably between about 0.2 and about 30% by weight, particularly preferably between about 0.4 and about 20% by weight, and in particular between about 0.8 and about 10% by weight of the enzyme protein.

The agents described herein comprise all conceivable types of washing or cleaning agents, both concentrates and also agents to be used in undiluted form, for use on a commercial scale, in the washing machine, or when washing or cleaning by hand. They include for example washing agents for textiles, carpets or natural fibers, for which the term washing agent is used. They also include for example dishwashing agents for dishwashers or manual dishwashing agents or cleaners for hard surfaces such as metal, glass, porcelain, ceramic, tiles, stone, painted surfaces, plastics, wood or leather, for which the term cleaning agent is used, that is to say, besides manual and automatic dishwashing agents for example, also scouring agents, glass cleaners, toilet cleaners, etc. The washing and cleaning agents in the context of the disclosure also include washing auxiliaries which are added to the actual washing agent in manual or automatic textile laundering in order to achieve a further effect. Furthermore, washing and cleaning agents in the context of the disclosure also include textile pre- and post-treatment agents, that is to say those agents with which the item of laundry is brought into contact prior to the actual laundering, for example in order to loosen stubborn stains, as well as agents which, in a step following the actual textile laundering, impart to the washed item further desirable properties such as a pleasant feel, absence of creases or low static charge. The last-mentioned agents include, inter alia, fabric softeners.

An agent described herein contains the peroxidase advantageously in an amount from about 2 μg to about 20 mg, preferably from about 5 μg to about 17.5 mg, particularly preferably from about 20 μg to about 15 mg, and very particularly preferably from about 50 μg to about 10 mg per g of the agent. Furthermore, the peroxidase contained in the agent, and/or further ingredients of the agent, can be encased with a substance that is impermeable to the enzyme at room temperature or in the absence of water, which substance becomes permeable to the enzyme under the use conditions of the agent. Such an embodiment of the disclosure is thus characterized in that the peroxidase is encased with a substance that is impermeable to the peroxidase at room temperature or in the absence of water. Furthermore, the washing or cleaning agent itself can also be packaged in a container, preferably an air-permeable container, from which it is released shortly before use or during the washing operation.

These embodiments of the present disclosure encompass all solid, powdered, liquid, gel-like or paste-like delivery forms of agents described herein, which optionally can consist of multiple phases and be present in compressed or uncompressed form. The agent may exist as a pourable powder, in particular with a bulk weight from about 300 g/l to about 1200 g/l, in particular about 500 g/l to about 900 g/l, or about 600 g/l to about 850 g/l. The solid delivery forms of the agent also include extrudates, granules, tablets or pouches. Alternatively, the agent may also be liquid, gel-like, or paste-like, for example in the form of a non-aqueous liquid laundry detergent or dishwashing detergent or a non-aqueous paste or in the form of an aqueous liquid laundry detergent or dishwashing detergent or a water-containing paste. The agent may also exist as a one-component system. Such agents consist of one phase. Alternatively, an agent can also consist of multiple phases. Such an agent is accordingly split into multiple components.

Very generally, the agent described herein can be prepackaged into dosage units. These dosage units preferably comprise the amount of substances with washing or cleaning activity that is required for one washing or cleaning operation.

The agents described herein, regardless of whether they are liquid or solid, in particular the premanufactured dosage units, particularly preferably have a water-soluble casing.

The water-soluble casing is preferably formed of a water-soluble film material which is selected from the group consisting of polymers or polymer mixtures. The casing may be formed of one or two or more layers of the water-soluble film material. The water-soluble film material of the first layer and of the further layers, if present, may be identical or different. Particular preference is given to films which can be glued and/or sealed to form packages, such as tubes or pods, after they have been filled with an agent.

It is preferred that the water-soluble casing contains polyvinyl alcohol or a polyvinyl alcohol copolymer. Water-soluble casings which contain polyvinyl alcohol or a polyvinyl alcohol copolymer have a good stability while having a sufficiently high solubility in water, in particular in cold water.

Suitable water-soluble films for producing the water-soluble casing are preferably based on a polyvinyl alcohol or a polyvinyl alcohol copolymer having a molecular weight in the range from about 10,000 to about 1,000,000 gmol⁻¹, preferably from about 20,000 to about 500,000 gmol⁻¹, particularly preferably from about 30,000 to about 100,000 gmol⁻¹, and in particular from about 40,000 to about 80,000 gmol⁻¹.

Polyvinyl alcohol is usually produced through the hydrolysis of polyvinyl acetate, since the direct synthesis route is not possible. The same applies to polyvinyl alcohol copolymers, which are correspondingly produced from polyvinyl acetate copolymers. It is preferred if at least one layer of the water-soluble casing comprises a polyvinyl alcohol having a degree of hydrolysis from about 70 to 100 mol %, preferably about 80 to about 90 mol %, particularly preferably about 81 to about 89 mol % and in particular about 82 to about 88 mol %.

A polyvinyl alcohol-containing film material suitable for producing the water-soluble casing may additionally have added to it a polymer selected from the group consisting of (meth)acrylic acid-containing (co)polymers, polyacrylamides, oxazoline polymers, polystyrene sulfonates, polyurethanes, polyesters, polyethers, polylactic acid or mixtures of the aforementioned polymers. Polylactic acids are a preferred additional polymer.

Preferred polyvinyl alcohol copolymers comprise, besides vinyl alcohol, also dicarboxylic acids as further monomers. Suitable dicarboxylic acids are itaconic acid, malonic acid, succinic acid and mixtures thereof, preference being given to itaconic acid.

Polyvinyl alcohol copolymers which are likewise preferred comprise, besides vinyl alcohol, also an ethylenically unsaturated carboxylic acid, a salt thereof, or an ester thereof. With particular preference, such polyvinyl alcohol copolymers contain, besides vinyl alcohol, also acrylic acid, methacrylic acid, acrylic acid ester, methacrylic acid ester, or mixtures thereof.

It may be preferred that the film material contains further additives. The film material may contain for example plasticizers such as dipropylene glycol, ethylene glycol, diethylene glycol, propylene glycol, glycerol, sorbitol, mannitol, or mixtures thereof. Further additives include for example release aids, fillers, crosslinking agents, surfactants, antioxidants, UV absorbers, antiblocking agents, non-stick agents, or mixtures thereof.

Suitable water-soluble films for use in the water-soluble casings of the water-soluble packages according to the disclosure are films which are sold by the company MonoSol LLC for example under the name M8630, C8400 or M8900. Other suitable films include films bearing the name Solublon® PT, Solublon® GA, Solublon® KC or Solublon® KL from Aicello Chemical Europe GmbH or the VF-HP films from Kuraray.

Washing or cleaning agents described herein may contain, in addition to the peroxidase described herein, also hydrolytic enzymes or other enzymes in a concentration useful for the efficacy of the agent. The enzymes may be present in the form of the enzyme formulations described above. A further embodiment of the disclosure is thus formed by agents that moreover comprise one or more further enzymes. As further enzymes, use can preferably be made of all enzymes which can display a catalytic activity in the agent described herein, in particular a protease, amylase, cellulase, hemicellulase, mannanase, tannase, xylanase, xanthanase, xyloglucanase, β-glucosidase, pectinase, carrageenase, perhydrolase, oxidase, oxidoreductase or a lipase, as well as mixtures thereof. Further enzymes are advantageously each contained in the agent in an amount from about 1×10⁻⁸ to about 5% by weight, based on active protein. With increasing preference, each further enzyme is contained in agents described herein in an amount from about 1×10⁻⁷ to about 3% by weight, from about 0.00001 to about 1% by weight, from about 0.00005 to about 0.5% by weight, from about 0.0001 to about 0.1% by weight, and particularly preferably from about 0.0001 to about 0.05% by weight, based on active protein.

The washing or cleaning agents described herein, which may exist as powdered solids, in compressed particle form, as homogeneous solutions or suspensions, may contain, besides a peroxidase described herein, also all known ingredients customary in such agents, wherein preferably at least one further ingredient is present in the agent. The agents described herein may in particular contain surfactants, builders, other bleaching agents or bleach activators. They may also contain water-miscible organic solvents, sequestering agents, electrolytes, pH regulators and/or further auxiliaries such as optical brighteners, graying inhibitors, foam regulators, as well as colorants and fragrances, and combinations thereof. In different embodiments of the disclosure, the agents described herein contain a hydrogen peroxide source, for example a percarbonate, peroxide or perborate. The hydrogen peroxide originating from this source can further increase the catalytic activity of the peroxidases described herein. However, it is preferred that the enzymes described herein can bring about an oxidative cleavage of carotenoids in the absence of hydrogen peroxide.

Advantageous ingredients of agents described herein are disclosed in the international patent application WO 2009/121725, starting on page 5, penultimate paragraph thereof, and ending on page 13 after the second paragraph. Reference is expressly made to this disclosure, and the disclosure content therein is incorporated into the present patent application.

A further subject matter of the disclosure is a method for cleaning textiles or hard surfaces which is characterized in that an agent described herein is used in at least one method step, or in that a peroxidase described herein becomes catalytically active in at least one method step, in particular in such a way that the peroxidase is used in an amount from about 40 μg to about 4 g, preferably from about 50 μg to about 3 g, particularly preferably from about 100 μg to about 2 g, and very particularly preferably from about 200 μg to about 1 g.

This includes both manual and automatic methods, preference being given to automatic methods. Methods for cleaning textiles are generally characterized in that, in multiple method steps, various substances having cleaning activity are applied onto the material to be cleaned and are washed out after the contact time, or in that the material to be cleaned is treated in some other way with a washing agent or a solution or a dilution of said agent. The same applies to methods for cleaning all materials other than textiles, particularly hard surfaces. All conceivable washing or cleaning methods can be supplemented, in at least one of the method steps, by the use of a washing or cleaning agent described herein or of a peroxidase described herein, and then represent embodiments of the present disclosure. All facts, subject matters and embodiments that are described for peroxidases described herein and agents containing them are also applicable to this subject matter of the disclosure. Reference is therefore expressly made at this point to the disclosure at the relevant point, with the indication that this disclosure also applies to the methods described above.

Embodiments of this subject matter of the disclosure are also formed by methods for treating textile raw materials or for textile care, in which a peroxidase described herein becomes active in at least one method step. Among these, preference is given to methods for textile raw materials, fibers or textiles having natural constituents, and very particular preference to those containing wool or silk.

A further subject matter of the disclosure is the use of an agent described herein for cleaning textiles or hard surfaces, or of a peroxidase described herein for cleaning textiles or hard surfaces, in particular such that the peroxidase is used in an amount from about 40 μg to about 4 g, preferably from about 50 μg to about 3 g, particularly preferably from about 100 μg to about 2 g, and very particularly preferably from about 200 μg to about 1 g.

All facts, subject matters and embodiments that are described for peroxidases described herein and agents containing them are also applicable to this subject matter of the disclosure. Reference is therefore expressly made at this point to the disclosure at the relevant point, with the indication that this disclosure also applies to the use described above.

### EXAMPLES

All molecular biology work steps follow standard methods as specified for example in the handbook by Fritsch, Sambrook and Maniatis “Molecular cloning: a laboratory manual”, Cold Spring Harbour Laboratory Press, New York, 1989, or comparable relevant works. Enzymes and kits were used according to the instructions of the particular manufacturer.

The chemicals used were of analytical purity and were obtained from Sigma-Aldrich (Munich), Carl Roth (Karlsruhe) or Merck (Darmstadt). The PCR primers were obtained from Eurofins MWG Operon (Ebersberg).

### Example 1: Culturing of Ganoderma applanatum

The Ganoderma applanatum (Gap) strain was obtained from CBS (Centraalbureau voor Schimmelcultures, Utrecht, The Netherlands). The cultures were plated onto standard nutrient liquid (SNL) agar plates containing 30 g l⁻¹ glucose monohydrate, 9 g l⁻¹ yeast extract, 4.5 g l⁻¹ L-asparagine monohydrate, 0.5 g l⁻¹ MgSO₄, 1.5 g l⁻¹ KH₂PO₄, 1 ml trace element solution (0.005 g l⁻¹ CuSO₄×5 H₂O, 0.08 g l⁻¹ FeCl₃×6 H₂O, 0.09 g l⁻¹ ZnSO₄×7 H₂O, 0.03 g l⁻¹ MnSO₄×H₂O, and 0.4 g l⁻¹ EDTA) and 15 g l⁻¹ agar agar, and were stored. The media were adjusted to pH 6.0 with 1 M NaOH.

To produce the pre-cultures, a piece of agar measuring 1 cm² containing a grown strain culture was cut from the agar plate, transferred into a 250 ml Erlenmeyer flask filled with 100 ml SNL (without agar), and homogenized. The pre-cultures were incubated at 150 rpm and 24° C. for 7 days. Then, 25 ml of the pre-culture were used to inoculate the main cultures (250 ml medium). These were incubated in SNL with 3 ml β-carotene emulsion (freshly prepared and sterile-filtered) at 24° C. and 150 rpm until the day of maximum extracellular β-carotene degradation activity (CD activity). The culture was then harvested, centrifuged at 5000 rpm and 4° C. (Rotina 380R, Hettich), and the cells were discarded. The active supernatant was then used for further purification.

### Example 2: Isolation of the Manganese Peroxidases from G. applatum

To isolate the peroxidase, the active supernatant of the fungal culture was carefully mixed 1:1 with a high salt buffer until a concentration of 2 M (NH₄)₂SO₄ (in 50 mM sodium phosphate, pH 6.5) was achieved. The precipitate was centrifuged (5000 rpm, 10 min), and the active supernatant was separated on a Phenyl Sepharose Fast Flow column (20 ml, GE Healthcare, Solingen). To this end, the sample was loaded onto the column at a flow rate of 2 ml min⁻¹, and the active enzyme was eluted by changing to 100% elution buffer (50 mM sodium phosphate, pH 6.5). The active fractions were desalinated by means of ultrafiltration and were concentrated. Thereafter, an anion exchange chromatography was carried out using a Q-Sepharose column (1 ml, GE Healthcare, Solingen) with 20 mM sodium acetate buffer pH 4.0 (+/−1 M sodium chloride). To this end, 1 ml of the sample (combined, desalinated and concentrated CD-active HIC fractions) was mixed with 10 ml salt-free running buffer and loaded onto the column. Separation was carried out at 1 ml min⁻¹ using a 3% stage (12 ml), followed by a linear gradient elution to 30% salt-containing buffer. The active fractions (˜10% NaCl-containing buffer) were once again concentrated by means of ultrafiltration and then fed onto a Superdex 75 gel filtration column (GE Healthcare, Solingen) and eluted at 0.5 ml min⁻¹ with buffer which contained 100 mM sodium phosphate and 100 mM sodium chloride (pH 6.5).

### Example 3: Enzyme Activity Assay

β-Carotene emulsion was mixed with buffer solution and distilled water to a concentration of 100 mM sodium acetate (pH 4.5) or sodium phosphate (pH 8.0) and an optical density (OD) of 1 at 450 nm. 270 μl of this substrate solution were pipetted into a 96-well plate, and the reaction was started by adding 30 μl of enzyme sample. The decrease in the extinction at 450 nm (-mAbs min⁻¹) was monitored for 20 min at 30° C. in a BioTek Synergy 2™ microplate reader.

For the β-carotene emulsion, 20 mg β-carotene and 1 g Tween 80 were dissolved in 20 ml dichloromethane. The solvent was then removed using a rotary evaporator (40° C., 800 mbar), and the emulsion was carefully mixed with 30 ml of distilled water, said water being at a temperature of 40° C. The remaining dichloromethane was removed at 40° C. while reducing the pressure in stages to 200 mbar. The emulsion was (0.45 μm) filtered into a 50 ml Erlenmeyer flask and the latter was topped up with warm water. The emulsion was stored in the dark for a maximum period of 2 weeks at 4° C.

In order to determine the pH dependency of the enzyme, Britton-Robinson buffer (phosphoric, acetic and boric acid, in each case 0.04 M were adjusted to different pH values using 1 M NaOH) was used in the range between pH 3 and 11.

The temperature dependency was measured in the range from 25 to 80° C. using a Shimadzu UV-VIS spectrophotometer (UV1650PC) equipped with a B. Braun Thermomixer (FRI60MIX). To this end, 720 μl substrate solution were heated for 5 minutes in a cuvette. 80 μl enzyme sample were then added in order to start the reaction. All measurements were carried out twice and measured against blank samples with buffer instead of enzyme.

The maximum β-carotene degradation activity of the peroxidases of SEQ ID Nos. 1 and 2 in the absence of H₂O₂ was observed at pH 4.5-5.0 and 45-55° C. A second, lower optimum was found at pH 8.0 with approximately 50% residual activity. Since commercial textile washing agents are alkaline, the enzyme activity at pH 8.0 is very interesting. Until now, no manganese peroxidases having a β-carotene degradation activity in the alkaline pH range were known. A comparison with other enzymes was therefore not possible.

Using the same method, the pH and temperature optimum was determined for the lignin peroxidase from Bjerkandera adusta (SEQ ID NO:3), which for this enzyme occurred at pH 10 and 20° C.

### Example 4: Mini Washing Test (Liquid Washing Agent, Tomato & Carrot)

The enzyme preparations were used in the following washing test:

Round punched-out soiled fabric specimens (diameter 1 cm) were placed individually in a 48-well microtiter plate (WfK 100 (cotton soiled with carrot juice) and WfK 10SG (cotton soiled with tomato beef sauce)).

A total of 1000 μl of solution was pipetted onto each piece of fabric, said solution being formed by a washing liquor preheated to 40° C. and consisting of a commercially available liquid washing agent (end concentration in the test 4.7 g/l, 16° dH) and of the enzyme solution to be tested, with the concentration specified below. The tests were carried out in triplicate.

The plates were closed in an air-permeable manner by the associated lid and were washed for 1 hour in the dark on a Titramax incubator shaker (600 rpm) at 40° C.

The washing liquor was then poured off through a screen, and rinsing was carried out three times with tap water and three times with deionized water; the remaining water was carefully drawn off by dabbing with lab soakers, and the fabric specimens were dried for 24 or 48 hours in the dark at room temperature. After the fabric specimens had been glued onto white paper, the lightness and color was measured using a Minolta colorimeter in comparison to the white and black standard of the device.

To evaluate the lightening, the lightness value L* in the L*a*b* system was used.

Table 1 shows the lightening for the manganese peroxidases from Ganoderma applanatum (culturing and isolation as described in Examples 1 and 2) having SEQ ID NO:1 and 2 (mixture of the isoforms) (higher values indicate greater lightening of the specimen):

Specimen 1: washing agent alone (reference)
Specimen 2: washing agent+enzyme solution concentration 1=0.13 mU/mL in the test
Specimen 3: washing agent+enzyme solution concentration 2=0.65 mU/mL in the test

TABLE 1

| Soil | Specimen 1 (reference) | Specimen 2 | Specimen 3 |
|---|---|---|---|
| Carrot juice | 92.5 | 93.3 | 93.4 |
| Tomato beef sauce | 81.6 | 84.0 | 86.9 |

A considerable lightening of up to 5.3 units could be seen especially in the case of tomato beef sauce. A significant change is deemed to be a change of 1 unit or more. Since carrot juice is already very light even with washing agent alone, no further significant lightening could be achieved in this case, but a clear tendency can be seen.

Table 2 shows the lightening for the lignin peroxidase from Bjerkandera adusta having SEQ ID NO:3 (enzyme overexpressed heterologously in E. coli and purified) (higher values indicate greater lightening of the specimen):

Specimen 1: washing agent alone (reference)
Specimen 2: washing agent+enzyme solution concentration 1=0.1 mU/mL in the test
Specimen 3: washing agent+enzyme solution concentration 2=0.2 mU/mL in the test

TABLE 2

| Soil | Specimen 1 | Specimen 2 | Specimen 3 |
|---|---|---|---|
| Tomato beef sauce | 81.0 | 84.9 | 85.7 |

A considerable lightening of up to 4.7 units could be seen in the case of tomato beef sauce. The more enzyme used, the greater the effect.

In order to determine the enzyme activity of the dye-decolorizing peroxidase from Bjerkandera adusta having SEQ ID NO:4 (culture isolated from Bjerkandera adusta), round punched-out soiled fabric specimens (diameter 1 cm) were placed individually (WfK 100 (cotton soiled with carrot juice) and WfK 10SG (cotton soiled with tomato beef sauce)).

A total of 3500 μl of solution was pipetted onto each piece of fabric, said solution being formed by a washing liquor preheated to 30° C. and consisting of a commercially available liquid washing agent without enzyme (Henkel AG, Düsseldorf) (end concentration in the test 0.44% by weight, 16° dH) and of a quantity of the enzyme solution to be tested which corresponded to a carotene degradation activity of −0.29 mU/mL.

The plates were closed in an air-permeable manner by the associated lid and were washed for 16 hours in the dark on a Titramax incubator shaker (150 rpm) at 30° C.

The washing liquor was then poured off through a screen, and rinsing was carried out three times with water; the remaining water was carefully drawn off by dabbing with lab soakers, and the fabric specimens were dried in the dark at 30° C. The quantitative degradation values were determined using 20 measurement points of an RGB color scanner against a blind specimen (without enzyme) (Table 3).

TABLE 3

| | | |
|---|---|---|
| R | G | B |

Tomato beef sauce

| | | | |
|---|---|---|---|
| Reference | 239 | 191 | 62 |
| Enzyme | 252 | 236 | 192 |

Carrot juice

| | | | |
|---|---|---|---|
| Reference | 243 | 238 | 205 |
| Enzyme | 248 | 244 | 214 |

The RGB values indicate a clear difference in color. If the RGB is converted into the lightness value L, the lightness values L* in the L*a*b* system are obtained as specified in Table 4:

Specimen 1: washing agent alone (reference)
Specimen 2: washing agent plus enzyme solution concentration 1=0.29 mU/mL

TABLE 4

| Soil | Specimen 1 | Specimen 2 |
|---|---|---|
| Tomato beef sauce | 653 | 701 |
| Carrot juice | 701 | 707 |

This means that a considerable lightening could be observed in the case of tomato beef sauce.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A peroxidase comprising an amino acid sequence that:
has a sequence identity of at least 60% to the amino acid sequence specified in SEQ ID NO:1 (Gap MnP1), across the entire length thereof; or
has a sequence identity of at least 60% to the amino acid sequence specified in SEQ ID NO:2 (Gap MnP2), across the entire length thereof; or
has a sequence identity of at least 80% to the amino acid sequence specified in SEQ ID NO:3 (Bja LiP), across the entire length thereof; or
has a sequence identity of at least 60% to the amino acid sequence specified in SEQ ID NO:4 (Bja DyP), across the entire length thereof.

2. (canceled)

3. (canceled)

4. (canceled)

5. A method for producing a peroxidase, said method comprising:
culturing a host cell which comprises a peroxidase according to claim 1; and
isolating the peroxidase from the culture medium or from the host cell.

6. An agent comprising at least one peroxidase, wherein the peroxidase comprises an amino acid sequence that:
has a sequence identity of at least 60% to the amino acid sequence specified in SEQ ID NO:1 (Gap MnP1), across the entire length thereof; or
has a sequence identity of at least 60% to the amino acid sequence specified in SEQ ID NO:2 (Gap MnP2), across the entire length thereof; or
has a sequence identity of at least 80% to the amino acid sequence specified in SEQ ID NO:3 (Bja LiP), across the entire length thereof; or
has a sequence identity of at least 60% to the amino acid sequence specified in SEQ ID NO:4 (Bja DyP), across the entire length thereof.

7. The agent according to claim 6, wherein
the peroxidase can be obtained from a peroxidase having the amino acid sequences specified in one of SEQ ID Nos. 1-4 as the starting molecule by single or multiple conservative amino acid substitution.

8. The agent according to claim 6, wherein the agent
is a laundry or dishwashing detergent.

9. The peroxidase according to claim 1 having a demonstrable activity for carotenoids as substrate.

10. (canceled)

11. The method according to claim 5 wherein the host cell secretes the peroxidase into the medium surrounding the host cell.

12. The agent according to claim 6, wherein the peroxidase can be obtained from a peroxidase having the amino acid sequence specified in one of SEQ ID Nos. 1-4 as the starting molecule by fragmentation, deletion mutagenesis, insertion mutagenesis or substitution mutagenesis and comprises an amino acid sequence that matches the starting molecule having the amino acid sequence according to one of SEQ ID Nos. 1-3 over a length of at least 100 contiguous amino acids.

13. The agent according to claim 6, wherein the peroxidase can be obtained from a peroxidase having the amino acid sequence specified in one of SEQ ID Nos. 1-4 as the starting molecule by fragmentation, deletion mutagenesis, insertion mutagenesis or substitution mutagenesis and comprises an amino acid sequence that matches the starting molecule having the amino acid sequence according to SEQ ID NO:4 over a length of at least 100 contiguous amino acids.

14. The agent according to claim 6, wherein:
the peroxidase can be obtained from a peroxidase having the amino acid sequences specified in one of SEQ ID Nos. 1-4 as the starting molecule by single or multiple conservative amino acid substitution; and
the peroxidase can be obtained from a peroxidase having the amino acid sequence specified in one of SEQ ID Nos. 1-4 as the starting molecule by fragmentation, deletion mutagenesis, insertion mutagenesis or substitution mutagenesis and comprises an amino acid sequence that matches the starting molecule having the amino acid sequence according to one of SEQ ID Nos. 1-3 over a length of at least 100 contiguous amino acids.

15. The agent according to claim 6, wherein:
the peroxidase can be obtained from a peroxidase having the amino acid sequences specified in one of SEQ ID Nos. 1-4 as the starting molecule by single or multiple conservative amino acid substitution; and
the peroxidase can be obtained from a peroxidase having the amino acid sequence specified in one of SEQ ID Nos. 1-4 as the starting molecule by fragmentation, deletion mutagenesis, insertion mutagenesis or substitution mutagenesis and comprises an amino acid sequence that matches the starting molecule having the amino acid sequence according to SEQ ID NO:4 over a length of at least 100 contiguous amino acids.

16. The agent according to claim 6, wherein the agent is a washing or cleaning agent.

17. The agent according to claim 6, wherein the agent additionally comprises a hydrogen peroxide source.

18. The agent according to claim 17, wherein the hydrogen peroxide source comprises a percarbonate, peroxide, perborate, or combinations thereof.

19. The agent according to claim 6, wherein the agent additionally comprises surfactants, builders, enzymes different from the peroxidase, bleaching agents, bleach activators, water-miscible organic solvents, sequestering agents, electrolytes, pH regulators, and/or further auxiliaries such as optical brighteners, graying inhibitors, foam regulators, as well as colorants and fragrances, and combinations thereof.

20. The agent according to claim 6, wherein the agent:
is a laundry or dishwashing detergent;
additionally comprises a hydrogen peroxide source; and
additionally comprises surfactants, builders, enzymes different from the peroxidase, bleaching agents, bleach activators, water-miscible organic solvents, sequestering agents, electrolytes, pH regulators, and/or further auxiliaries such as optical brighteners, graying inhibitors, foam regulators, as well as colorants and fragrances, and combinations thereof.

21. The agent according to claim 6, utilized for cleaning textiles or hard surfaces.
